(19) United States
(12) Patent Application Publication
(10) Pub. No.: US 2012/0101519 A1
(43) Pub. Date: Apr. 26, 2012

(54) POROUS VASCULAR CLOSURE PLUG WITH STARCH POWDER

(75) Inventors: Jason P. Hill, Brooklyn Park, MN (US); Mark L. Jenson, Greenfield, MN (US); Haitao Zhang, Maple Grove, MN (US); Kai Wang, Plymouth, MN (US); Pu Zhou, Maple Grove, MN (US)

(73) Assignee: BOSTON SCIENTIFIC SCIMED, INC., Maple Grove, MN (US)

(21) Appl. No.: 13/278,853

(22) Filed: Oct. 21, 2011

Related U.S. Application Data
(60) Provisional application No. 61/406,412, filed on Oct. 25, 2010.

(51) Int. Cl. A61B 17/03

(52) U.S. Cl. 606/213

(57) ABSTRACT

The disclosure provides a composite plug for vascular closure including a hemostatic material, and a method of manufacturing the composite plug including a hemostatic material. The composite plug may comprise one or more materials having varying porosity, density, or composition, and may include a powdered hemostatic material.

### RELATED APPLICATIONS

This application claims priority to U.S. Provisional Application Ser. No. 61/406,412 filed Oct. 25, 2010.

### BACKGROUND

A large number of diagnostic and interventional procedures involve the percutaneous introduction of instrumentation into a vein or artery. For example, coronary or peripheral angioplasty, angiography, atherectomy, stenting of arteries, and many other procedures often involve accessing the vasculature through a catheter placed in the femoral artery or other blood vessel. Once the procedure is completed and the catheter or other instrumentation is removed, bleeding from the punctured artery must be controlled.

Traditionally, external pressure is applied to the skin entry site to stem bleeding from a puncture wound in a blood vessel. Pressure is continued until hemostasis has occurred at the puncture site. In some instances, pressure must be applied for up to an hour or more during which time the patient is uncomfortably immobilized. If blood activated clotting time (ACT) is elevated, due to the use of anticoagulants for example, a waiting period of up to an hour may be required to allow the ACT value to return to a normal or moderately-elevated level (70-200 seconds, for example), before a sheath or other medical device is removed and hemostasis attempted by manual compression. Further, external pressure to close the vascular puncture site works best when the vessel is close to the skin surface but may be unsuitable for patients with substantial amounts of subcutaneous adipose tissue since the skin surface may be a considerable distance from the vascular puncture site.

There are several approaches to close the vascular puncture site including the use of anchor and plug systems as well as metal clip systems and suture systems. Internal suturing of the blood vessel puncture requires a specially designed suturing device. These suturing devices involve a significant number of steps to perform suturing and require substantial expertise. Additionally, when releasing hemostasis material at the puncture site and withdrawing other devices out of the tissue tract, the currently employed approaches to sealing the puncture may only partially occlude the tract thereby allowing blood to seep out of the puncture.

### SUMMARY

In one aspect, the disclosure pertains to a composite plug comprising a porous biodegradable material. The porous biodegradable material may comprise a low-profile fabric or woven structure with sufficient space between the fibers to allow a substantial amount of a hemostatic material to be disposed within the pore spaces between the fibers. The hemostatic material and the porous biodegradable material may provide improved hemostasis with a reduced plug volume. A composite plug having a hemostatic material combined with the porous biodegradable material may provide an increased rate of bio-absorption.

In another aspect, the disclosure pertains to a composite plug for vascular closure comprising collagen, gelatin, or other porous and/or fibrous, biodegradable material in a generally cylindrical structure. The plug may comprise a foam, sponge, or other similar construction. The composite plug may comprise enlarged pores and/or porous structure with a hemostatic material disposed within the pores and/or porous structure for improved hemostasis effectiveness. The composite plug may comprise a core member having a lumen connecting a distal end and a proximal end, said lumen sized to receive a suture.

In another aspect, the disclosure pertains to a method of manufacturing a composite plug comprising the steps of obtaining a fibrous collagen or gelatin material; fabricating a low-profile fabric or woven structure; and disposing a starch powder within the spaces of the low-profile fabric or woven structure. The method may further comprise compressing the vascular plug to obtain a desired structure or dimension prior to use.

In another aspect, the disclosure pertains to a method of manufacturing a composite plug for vascular closure comprising the steps of obtaining a porous foam blank larger than a desired core member; removing excess foam from the porous foam blank larger than a desired core member to form a core member having a distal end and a proximal end; providing a lumen sized to receive a suture, said lumen connecting the distal end and the proximal end of core member; disposing a hemostatic material within the porous structure of the porous foam; providing a suture within the lumen which extends distally and proximally from the core member; and partially compacting the plug.

### BRIEF DESCRIPTION OF DRAWINGS

FIG. 1A is a perspective view of a composite plug.

FIG. 1B is a perspective view of a composite plug.

FIG. 2A is a perspective view of a composite plug having a core member and an outer member.

FIG. 2B is a perspective view of a longitudinally sectioned composite plug of FIG. 2A.

FIG. 3 is a perspective view of a composite plug.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

Embodiments are described herein in the context of a vascular closure plug. Those of ordinary skill in the art will appreciate that the following detailed description is illustrative only and is not intended to be in any way limiting. In the interest of clarity, not all of the routine features of the implementations described herein are shown and described.

Providing hemostasis at a blood vessel puncture site is important for procedures such as percutaneous access to prevent bleeding and hematoma. Thus, a solution to facilitate hemostasis at a puncture site may be achieved by deploying a composite vascular closure plug within the puncture tract.

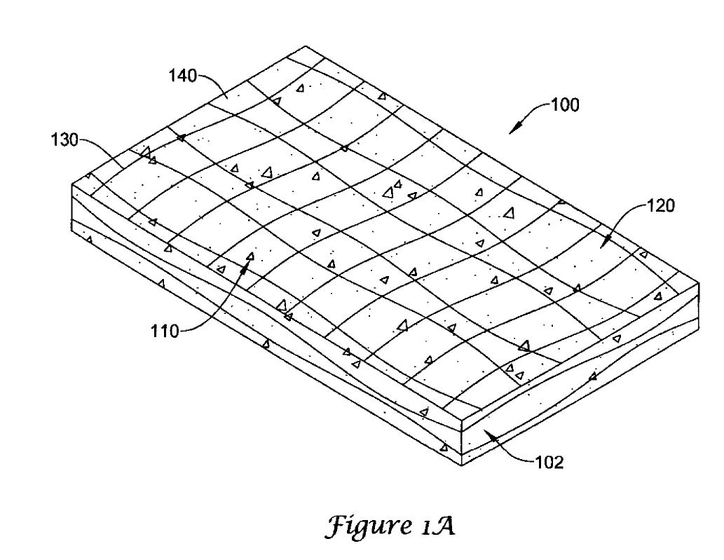

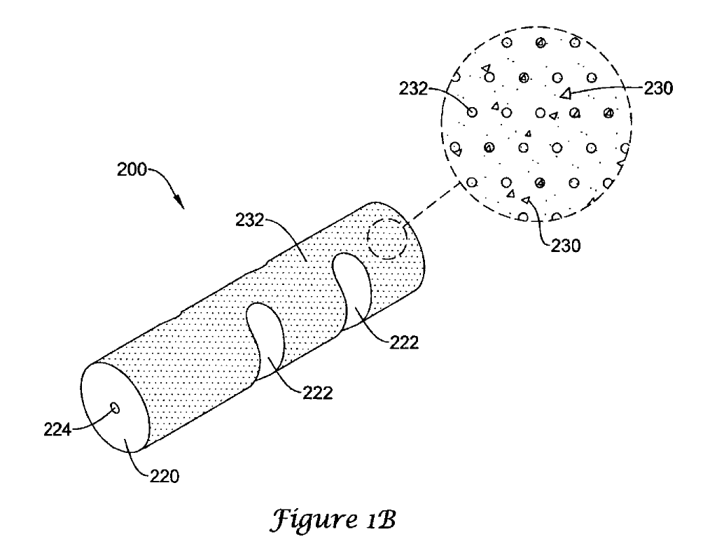

FIG. 1A illustrates an exemplary composite plug (100) having a low-profile construction. Composite plug (100) may be formed as a fabric, a felt, a mat, a strip, or a knitted or woven structure. Composite plug (100) may comprise a biodegradable plug material (102) such as collagen, gelatin, fibrin, cellulose, glycols, polysaccharides, protein-based analogs, polyurethane, polyesters, or other natural or synthetic materials. Composite plug (100) may comprise individual fibers (130) knitted, woven, or otherwise assembled together, or fibrous biodegradable materials contained in a suitable substrate. The plug material (102) may be a porous substrate comprising, for example, processed bulk tissue rendered porous by mechanical or chemical means, a foam or sponge made of tissue-origin or synthetic analog materials, or protein materials formed into fibers, and used to construct a mat, a fabric, or a knitted or woven structure. In some embodiments, composite plug (100) may have one or more apertures (not shown) sized and configured to pass a suture (not shown) therethrough. A suture (not shown) used with composite plug (100) may extend through a single lumen or may extend through a plurality of apertures (not shown) and/or lumen segments to aid in compression of composite plug (100).

A knitted or woven structure, such as a fabric, may be fabricated with sufficient space between the individual fibers (130) to allow a substantial amount of a hemostatic material such as starch material (110), wherein the starch material is a powder, spray, gel, or other form, to be applied. The starch material (110) may be disposed on an outer surface (140) of the structure, or may reach some distance from the outer surface (140) of the structure to be disposed within the structure itself or within the spaces or pores (120) between the fibers (130). Alternatively, the starch material (110) may be disposed in any suitable combination of locations.

Starch material (110) may comprise a commonly available starch such as BleedArrest™ Clotting Powder (Hemostasis, LLC, St. Paul, Minn.), PerClot™ (Starch Medical, San Jose, Calif.), SuperClot™ (Starch Medical, San Jose, Calif.), Vivastar™ (JRS PHarma GmbH+Co. KG, Rosenberg, Germany), Arista™ AH (Medafor, Minneapolis, Minn.), or others, and may be employed alone or in combination with polyethylene glycol (PEG) as a binder. Starch material (110) may be mixed with the binder or may be adhered to the plug material (102) by contacting the plug material (102) with the starch material (110) under conditions in which one or both have moistened surfaces. For example, a hydrogel carrier material may be applied from solution by using a number of techniques, for example, spraying, and when the carrier material has partially evaporated to a tacky state, the starch material (110) may be applied to the carrier material by impingement or by tumbling, for example. In some embodiments, the starch material (110) may be applied directly to the plug material (102) where it may be mechanically retained within the pores (120).

Composite plug (100) may comprise approximately equal amounts of starch material (110) and plug material (102) by weight. Composite plug (100) may also comprise an increased amount of starch material (110), such that the starch material (110) makes up about 60%, about 70%, about 80%, or up to about 90% of the total weight of the composite plug (100). The amount of starch material (110) as a percentage of total weight of the composite plug (100) may vary in or near an appropriate range in accordance with the disclosure herein. That is, the amount of starch material (110) may comprise a range of about 60% to about 90% of the total weight of the composite plug (100), or may comprise a different range therein, such as about 70% to about 80%, about 60% to about 75%, about 65% to about 85%, about 75% to about 90% of the total weight, or other appropriate combination or portion of a range within the scope of the disclosure. Accordingly, the composite plug (100) may comprise a ratio of starch material (110) to plug material (102) that is about 1:1, about 2:1, about 3:1, about 4:1, about 5:1, about 6:1, about 7:1, about 8:1, about 9:1, about 10:1, or other suitable ratio, including fractional or decimal amounts subsumed within the disclosed range, such as, for example, about 2.3:1, about 2.50:1, about 2.75:1, or other suitable ratios.

The amount of starch material (110) that may be added to the plug material (102) may depend upon one or more of several factors, such as, but not limited to, the bulk density of the porous substrate, the exposed surface area, pore openness and/or interconnectivity, and pore size relative to starch particle size. For example, deposits of starch material (110) on or in a relatively dense substrate may be limited to the exposed surface. A substrate with low bulk density, interconnected pores, and/or large pore size relative to starch particle size may be more heavily loaded throughout. The level of starch loading may be limited or controlled by the axial compression stiffness of the composite plug (100), which may control the plug's ability to collapse during deployment. Axial compression stiffness may be a function of, for example, loaded substrate bulk density, axial compression ratio, and/or geometry (such as a design or feature that may aid or control buckling, for example).

When placed at an arteriotomy site, blood will enter the pores (120), causing the starch material (110) and the plug material (102) to absorb fluid and swell. The plug material (102) may provide overall cohesive strength to the structure, and may provide some mechanical swelling or pro-clotting function. On the other hand, the starch material (110) can absorb water very readily and can quickly form a starch paste. The start paste can improve the plug material's (102) ability to stop the flow of blood at the arteriotomy site by providing additional structural support to the porous plug material (102), mechanically blocking the pores (120) to reduce leakage through the porous structure, and by causing blood to clot in the pores (120) of the composite plug (100) and adjacent to the composite plug (100) to provide improved hemostasis compared to composite plugs without starch material (110).

FIG. 1B illustrates an exemplary composite plug (200) for vascular closure comprising collagen, gelatin, or other porous biodegradable material in a generally cylindrical structure, In some embodiments, composite plug (200) may have an elongate core member (220) provided with a lumen (224) extending axially from a distal end to a proximal end and sized to accept a suture (not shown). However, the lumen (224) need not extend completely from the proximal end to the distal end of the core member (220), and may extend through a portion of core member (220). In some embodiments, core member (220) may be provided with a plurality of apertures (not shown) and/or lumen segments sized to accept a suture. The core member (220) may be formed from porous foam or sponge of collagen or gelatin such as GELFOAM® (Pharmacia & Upjohn, Inc., Bridgewater, N.J.), Surgifoam™ (Johnson & Johnson, New Brunswick, N.J.), CURASPON® (CuraMedical BV, Assendelft, Netherlands), GELITASPON® (Gelita Medical BV, Amsterdam, Netherlands), GELASPON® (Juvalis—Bernburg, Germany), or other suitable materials such as, but not limited to, those materials described herein.

The composite plug (200) may comprise enlarged pores (232) and/or a porous structure with a material (230) at least partially disposed within the pores (232) and/or porous structure for improved hemostasis effectiveness. Material (230) may include a hydrogel, a hemostatic material, an antimicrobial, a growth promoter, thrombus enhancing agents, and the like. The hydrogel component, if present, may include known biocompatible hydrogels, such as polyethylene glycols (PEG) on the molecular weight range of about 600 to 6,000, including PEG 900, PEG 3350, and PEG 6000 (Sigma-Aldrich, St. Louis, Mo.). Of the hemostatic materials commonly available, starch such as BleedArrest™ Clotting Powder (Hemostasis, LLC, St. Paul, Minn.), PerClot™ (Starch Medical, San Jose, Calif.), SuperClot™ (Starch Medical, San Jose, Calif.) or Arista™ AH (Medafor, Minneapolis, Minn.) may be employed alone or in combination with polyethylene glycol as a binder. As discussed herein, the hemostatic material may be mixed with the binder or may be adhered to the plug component(s) by contacting the plug component with the hemostatic material under conditions in which one or both have moistened surfaces. In some embodiments, the hemostatic material may be applied directly to plug (200) where it may be mechanically retained within the pores. In addition to serving as a binder for a hemostatic material, the hydrogel component may also be used to modulate the rate of swelling of the porous structure and, in some embodiments, may serve as a lubricant during the deployment of the composite plug.

The composite plug (200) of FIG. 1B may optionally include horizontal or vertical slits, notches, grooves, helical grooves, and the like which may facilitate axial compression of the plug and may provide controlled buckling and folding of the plug. Notches (222) are illustrated in FIG. 1B.

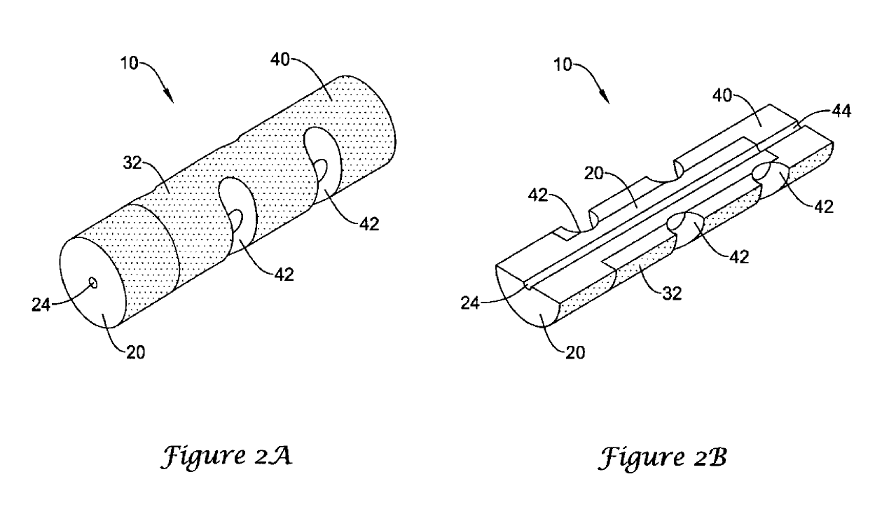

FIGS. 2A-B, which together illustrate an alternative composite plug (10) having an elongate core member (20) provided with a lumen (24), additionally include an outer member (40) having the general shape of a cylindrical cap which mates with a core member (20) having a T-shaped axial cross-section, such that composite plug (10) resembles a generally cylindrical distal segment joined to a generally cylindrical proximal segment. Either of core member (20) and outer member (40) may have, or alternatively both may have, a porous structure including a plurality of pores (32). The outer member (40) has at least a first lumen which may receive at least a portion of the core member (20) and typically has a second lumen (44) sized and aligned with the lumen (24) of the core member (20) to receive the suture (not shown) extending through core member (20) to provide a continuous path therefor. The exterior surfaces of core member (20) distal segment and the outer member (40) proximal end may have substantially the same cross-section to provide a smooth joint between core member (20) and the outer member (40). An outer member lumen may be sized and positioned to accept at least a portion of the core member (20) therein either before or after partial radial compression of that portion of the core member (20) to be contained within the first lumen of the outer member (40).

Core member (20) may have a generally T-shaped axial cross-section and generally circular transverse cross-sections. Outer member (40) for a core member (20) having a generally T-shaped axial cross-section may have a mating generally U-shaped axial cross-section. In such embodiments, the cross-section of the enlarged distal end of core member (20) may be similar to the cross-section of the distal end of outer member (40) to provide a smooth transition between the two members. In some embodiments, outer member (40) may extend a short distance proximal of the proximal end of core member (20). In these and other embodiments, the distal end of core member (20) may extend a short distance distal of the distal end of outer member (40).

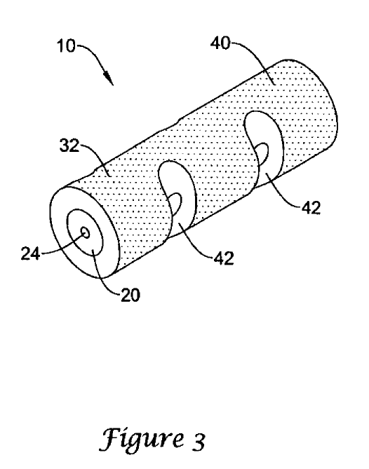

Alternatively, core member (20) and outer member (40) need not assume a T-shaped axial cross-section and cap configuration. Core member (20) and outer member (40) may be arranged as a core member with an outer member coaxially disposed about the core member, such as shown in FIG. 3. In some embodiments, the core member (20) and the outer member (40) may have different overall lengths. For example, the core member (20) may extend from the proximal end to the distal end of composite plug (10), but the outer member (40) may not extend from the proximal end to the distal end of the composite plug (10), or the outer member (40) may be shorter in length than the core member (20). In some embodiments, when the composite plug (10) is axially compressed, the core member (20) may compress or collapse at a different rate than the outer member (40).

It will be appreciated that transverse cross-sections of the core member(s) and the outer member(s) are not necessarily circular and that the overall shape of the composite plug is not necessarily cylindrical. For example, both the core member (20) and the outer member (40) may have a square, rectangular, or other suitable cross-section or shape. In an embodiment having a non-circular cross-section, it may be possible for the composite plug (10) to assume a generally round or circular cross-section following radial compression.

Core member (20) and outer member (40) may each be formed using the same plug material, a different plug material, or the same plug material but having a different density or porosity. Core member (20) and outer member (40) may be formed either with or without an added hemostatic material as described herein. For example, core member 20 may comprise a collagen or gelatin plug, while outer member (40) may comprise a composite plug similar to the exemplary plugs described herein having an added hemostatic material. Alternatively, outer member (40) may comprise a collagen or gelatin plug while core member (20) comprises a composite plug having an added hemostatic material, or both the core member (20) and the outer member (40) may comprise a composite plug having an added hemostatic material.

Alternatively, plug (10) may comprise a core member (20) having a density that is higher than the density of an outer member (40), or vice versa. Differing densities may permit the different members of plug (10) to retain different characteristics that may be beneficial during deployment. Deployment may typically involve compression of plug (10) at a blood vessel puncture site, in some cases along a suture or guidewire, with a knot, a cinching element, or other holding element used to maintain the plug (10) in a compressed state by resisting the expansion of the plug. A higher density material may have an increased resistance to migration over the knot or cinching element when being hydrated and/or deployed when compared to a lower density material, which may begin to flow back over the knot, cinching element, or other holding element due to the plug's natural tendency to expand. A lower density material may have an increased resistance to fracture during deployment compared to a higher density material. Accordingly, during deployment, a plug (10) comprising a higher density core member (20) and a lower density outer member (40) disposed about the core member (20) may comprise the mutually beneficial characteristics of increased resistance to migration and increased resistance to fracture.

As in the monolithic plug of FIGS. 1A-B, the plug of FIGS. 2A-B may optionally include horizontal or vertical slits, notches, grooves, helical grooves, and the like which may facilitate axial compression of the plug and to provide controlled buckling and folding of the plug. Notches (42) are illustrated. Additionally, slits, notches, grooves, or other surface modifications may provide access through the outer member (40) to the core member (20) for improved fluid absorption by the core member (20) and/or the hemostatic material. Suitable modifications to control buckling of the composite plugs of this disclosure may be found, for example in co-pending applications Ser. No. 12/389,960, filed Feb. 20, 2009 and Ser. No. 12/390,067, filed Feb. 20, 2009 incorporated herein by reference in their entirety.

The components and placement of the hemostatic material may be selected to enhance or to impede the uptake of fluid by a composite plug, such as those composite plugs described herein, and so may be used to control the intermediate shapes which the composite plug adopts as it swells locally in response to fluid contact. For example, partially filling the porous structure of a collagen or gelatin foam with a hemostatic material such as a hydrogel. Hydrogel, particularly a higher molecular weight hydrogel, will often impede uptake of fluid and so will delay the swelling of the composite plug locally. Conversely, lower molecular weight hydrogels may serve as wetting agents or surfactants and may enhance the uptake of fluids thereby accelerating swelling. Alternatively, a hemostatic material such as starch powder that is disposed within the porous structure of a composite plug may swell rapidly on contact with fluid so as to mechanically block the pores to reduce fluid leakage and enhance clot formation.

Although it is within the scope of the disclosure to apply the hemostatic material by any method known in the art, impingement, dip coating, and spray coating have been found to be appropriate and convenient for some materials. For example, a hemostatic material such as hydrogel may be applied from solutions in water, isopropanol, and ethanol. Polyethylene glycol (PEG) applied to a collagen or gelatin foam prior to radial compression has been found to increase the volumetric expansion upon hydration. In some embodiments, it may be desirable to distribute PEG, or other hydrogel material, throughout a collagen or gelatin foam by applying a hydrogel from an alcoholic solution by dip coating. The distribution of PEG within the foam may be controlled by varying the temperature and dip time. The resulting distribution may be essentially uniform or may gradually increase in concentration from the center toward the surface. In some embodiments, the distribution may vary in concentration in different areas of the foam. Addition of suitable surfactants may increase the rate of penetration of PEG solutions through a collagen or gelatin foam. Drying of the plug following dip coating may be accomplished with or without the application of vacuum. The drying process may influence shrinkage and/or hydration volume expansion capacity without significantly affecting leak performance.

In addition to providing a hemostatic material on a portion of the surface of a core member, such as core member (20) and/or outer member, such as outer member (40), a composite plug such as those described herein may be further modified by local mechanical compression or elongation, partially collapsing the plug material using heat, exposure of the surface to water followed by drying, or the like, to reduce the size of the pores and provide increased retention of the hemostatic material within the composite plug. Although the dimensions of the composite plug may be varied depending on anticipated usage sites, the length of the composite plug will often be greater than the average diameter or thickness of the plug and advantageously may be selected to be about four to five times the average diameter or about four to about ten or more times the average thickness prior to any compression, although greater or lesser ratios may be employed. The composite plug may be rolled, pressed, squeezed, or otherwise compressed to reduce the radial size of a cylindrical-shape plug, to reduce the thickness of low-profile knitted or woven plug, to reduce the size of the pores within the structure, and/or to facilitate fabrication or manufacturing of a multi-component plug. An increased pore size during fabrication may allow easier and superior penetration of the hemostatic material into the porous structure of the composite plug. After application of the hemostatic material, the porous plug may be compressed to obtain a desired structure and dimension prior to use.

A composite plug, such as those described above, may be formed, for example, by obtaining a fibrous collagen or gelatin plug material. A low-profile, porous fabric or woven structure may be fabricated using the fibrous plug material, such that the structure includes spaces or pores between the individual fibers of the plug material. A hemostatic material, such as a starch powder, may be applied to the plug material. The hemostatic material may be applied within the spaces or pores of the low-profile fabric or woven structure. The spaces or pores may have an enlarged size prior to disposing the hemostatic material within the spaces or pores for easier and superior penetration of the hemostatic material into the porous structure of the composite plug. After application of the hemostatic material, the composite plug may be at least partially compressed to obtain a desired structure and/or dimension prior to use. Compression of the composite plug may improve mechanical retention of the hemostatic material within the porous structure.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A composite plug for vascular closure comprising:
a biodegradable material substrate; and
a fast-acting hemostatic agent disposed on the substrate;
wherein the composite plug is expandable from a low-profile introduction configuration to a larger-dimension sealing configuration when implanted at an arteriotomy;
wherein the fast-acting hemostatic agent is configured to absorb fluid and swell upon contact with fluid;
wherein the composite plug is configured to allow passage of a cinching filament through at least a portion of the composite plug.

2. The composite plug of claim 1, wherein the biodegradable substrate includes collagen or gelatin.

3. The composite plug of claim 1, wherein the fast-acting hemostatic agent includes starch powder.

4. The composite plug of claim 1, wherein the biodegradable substrate is porous.

5. A composite plug for vascular closure comprising:
a fibrous biodegradable material having a low-profile woven structure and a plurality of spaces formed between individual fibers of the woven structure; and
a hemostatic material comprising a starch material at least partially disposed within the plurality of spaces;
wherein the plug has been partially compacted such that the hemostatic material at least partially disposed within the plurality of spaces is mechanically retained within the plurality of spaces.

6. The composite plug of claim 5, wherein the composite plug comprises a total weight, wherein the biodegradable material and the hemostatic material comprise about equal amounts of the total weight of the composite plug.

7. The composite plug of claim 5, wherein the composite plug comprises a total weight, wherein the hemostatic material comprises about 60% to about 90% of the total weight of the composite plug.

8. The composite plug of claim 5, wherein the starch material forms a starch paste upon contact with water, the starch paste mechanically blocking the plurality of spaces.

9. The composite plug of claim 5, wherein the low-profile woven structure has a length, the length being about four to about ten times an average thickness of the low-profile woven structure prior to compacting.

10. The composite plug of claim 5, where compaction of the composite plug reduces the size of the plurality of spaces.

11. A composite plug for vascular closure comprising:
an elongate cylindrical core member having a distal end, a proximal end, and a lumen connecting the distal end and the proximal end, the lumen sized to receive a suture, the core member including a porous structure formed of a biodegradable protein-derived biological material; and
a fluid-absorbing hemostatic material comprising a starch material;
wherein the hemostatic material is disposed on an outer surface of the core member and is at least partially disposed within the porous structure;
wherein the plug has been partially compacted in a radial dimension.

12. The composite plug of claim 11, further comprising one or more regions in which a portion of the plug has been removed.

13. The composite plug of claim 12, wherein the one or more regions in which a portion of the plug has been removed is in the form of a notch or a groove.

14. The composite plug of claim 12, wherein the starch material is in powdered form.

15. The composite plug of claim 11, wherein the core member has been modified by the inclusion of one or more slits.

16. The composite plug of claim 11, further comprising an outer member at least partially surrounding the core member, the outer member having a distal end, a proximal end, and a lumen connecting the distal end and the proximal end;
wherein the lumen of the outer member is sized to receive a suture and is axially aligned with the lumen of the core member.

17. The composite plug of claim 16, wherein the outer member comprises a biodegradable porous structure.

18. The composite plug of claim 17, wherein the outer member further comprises a starch material disposed on an outer surface of the outer member and at least partially disposed within the porous structure.

19. The composite plug of claim 16, wherein a portion of the core member extends distally with respect to the outer member.

20. The composite plug of claim 19, wherein the distal end of the outer member and the portion of the core member which extends distally with respect to the outer member have substantially the same cross-section.
